(19) United States
(12) Patent Application Publication
(10) Pub. No.: US 2024/0392088 A1
(43) Pub. Date: Nov. 28, 2024

(54) BINDING SUPPORT AND USES THEREOF

(71) Applicant: ANTEO TECHNOLOGIES PTY LTD, Eight Mile Plains, Queensland (AU)

(72) Inventors: Joe Maeji NOBUYOSHI, Newmarket, Queensland (AU); Chang-Yi HUANG, Calamvale, Queensland (AU); Manuel Christoph WIESER, Rochedale South, Queensland (AU); Kai-Anders HANSEN, Runcorn, Queensland (AU)

(73) Assignee: ANTEO TECHNOLOGIES PTY LTD, Eight Mile Plains, Queensland (AU)

(21) Appl. No.: 18/696,890

(22) PCT Filed: Oct. 7, 2022
(86) PCT No.: PCT/AU2022/051211

§ 371 Date: Mar. 28, 2024

(30) Foreign Application Priority Data
Oct. 8, 2021 (AU) 2021903232

(51) Int. Cl. C08J 9/00; G01N 33/543; G01N 33/553

(52) U.S. Cl. CPC C08J 9/0052; G01N 33/54346; G01N 33/54353; G01N 33/54388; G01N 33/553; C08J 2207/10; C08J 2301/18

(57) ABSTRACT

The disclosure generally relates to improving affinity interactions by the formation of an interconnected affinity agent network within a porous substrate via the use of metal complexes. More particularly, the disclosure generally relates to a binding support comprising a porous substrate as a component of a lateral flow assay device comprising at least one affinity agent associated with at least one metal coordination complex which forms an at least partially interconnected network with the porous substrate.

### FIELD OF THE INVENTION

The disclosure relates to, inter alia, improving affinity interactions by the formation of an interconnected affinity agent network within a porous substrate via the use of metal complexes. In one aspect, the disclosure relates to a binding support comprising a porous substrate as a component of a lateral flow assay device comprising at least one affinity agent associated with at least one metal coordination complex which forms an at least partially interconnected network with the porous substrate.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Any antibody/antigen interaction will have a specific equilibrium dissociation constant (K_{D}) defined by the ratio of their dissociation (off) and association (on) constants. K_{D} and affinity are therefore inversely related. In an ideal assay, K_{D} determines the equilibrium and, in the commercially important area of early detection when analyte is in low abundance, this equilibrium takes a not insignificant amount of time to reach. When the antibody or antigen is bound to a solid particle or a membrane, as is commonly the case in immunoassays, the loss of mobility means that this equilibrium takes even longer to be achieved.

Laboratory-based tests, by using automated mixing, washing, temperature regulation, and the like, can improve kinetics and help maximise reproducibility, sensitivity and specificity. Lateral flow, and other rapid tests, however are typically limited by sample volume and they therefore cannot be agitated or utilise multiple washing steps, and are generally limited by a maximum test result time of around 20 minutes. Assuming higher affinity capture agents cannot be sourced, the only remaining options are to somehow improve the mobility, accessibility and/or increase the number of capture agents per unit surface area. Many different approaches have been investigated but all have their drawbacks or limitations.

Simply maximising the density of capture agents per unit surface area does not necessarily improve performance. It has been demonstrated that non-saturated conditions may mitigate against overcrowding and improve accessibility for capture agent/analyte binding. In conjunction, improving the capture agent orientation may also further improve performance. In one approach described in US 2008/0176340A1, biotin or a biotin specific binder such as streptavidin is first bound to a surface, and then either biotinylated or streptavidin conjugated capture agent is added allowing for a degree of improved mobility and accessibility. This disclosure emphasises that saturating or maximising the density of capture agent per unit surface area is not always preferred.

Other approaches have sought to maximise the density of capture agents by coating the substrate surface with polymeric forms of streptavidin or analogues thereof. Compared with the corresponding monomeric forms these polymeric versions improved binding capacity for biotinylated capture agents as described in U.S. Pat. No. 6,638,728.

WO2020/089678 provides organic nanostructured molecules which contain linear regions that can covalently couple multiple capture agents. These organic nanostructured molecules are non-covalently bound to the surface of the membrane in an attempt to provide improved reproducibility, reliability, and selectivity. In this approach the capture agent can be covalently coupled to these organic nanostructured molecules before or after these nanostructured molecules are printed onto the membrane.

Such approaches may provide a degree of confirmation of the theoretical expectations of antibody/antigen interactions on these materials but they are not generally applicable practical solutions. For example, if the capture agent is covalently coupled to an organic nanostructured molecule before immobilisation to a membrane it requires the synthesis and purification of precursor molecules for each capture agent. In the screening for capture agent in assay development and/or manufacturing of a particular immunoassay product, a capture agent precursor must therefore be synthesised and this will require significant chemistry development not normally associated with immunoassay developers. Reproducibility of assay outcome may well be affected by precursor synthesis and non-covalent binding to the membrane affected by the presence of capture agent. Such complexities may be avoided if the organic nanostructured molecule is first immobilised onto a membrane. While this will provide a common membrane precursor, it requires coupling of capture agents onto membranes via covalent chemistry, with all its associated challenges.

The present disclosure may address or mitigate one or more of the aforementioned shortcomings or offer a useful commercial alternative.

### SUMMARY OF THE INVENTION

In a first aspect of the disclosure, there is provided a method of forming a binding support, the binding support comprising a polymeric porous substrate formed from at least one polymer; an oligomeric metal coordination complex associated with the at least one polymer forming the polymeric porous substrate; and at least one affinity agent associated with the oligomeric metal coordination complex, the method including the steps of:
(a) providing a polymeric porous substrate formed from at least one polymer;
(b) providing a liquid formulation comprising an oligomeric metal coordination complex associated with at least one affinity agent, within a liquid carrier; and
(c) contacting the polymeric porous substrate with the liquid formulation, to thereby form the binding support.

In a second aspect of the disclosure, although not necessarily the broadest aspect, there is provided a binding support comprising:
(a) a polymeric porous substrate formed from at least one polymer;
(b) an oligomeric metal coordination complex associated with the at least one polymer forming the polymeric porous substrate; and
(c) at least one affinity agent associated with the oligomeric metal coordination complex.

In embodiments, the binding support is a component of an assay device.

In a third aspect of the disclosure, there is provided a method of capturing a target molecule from a sample including the steps of:
(a) providing a binding support comprising a polymeric porous substrate formed from at least one polymer; an oligomeric metal coordination complex associated with the at least one polymer forming the polymeric porous substrate; and at least one affinity agent specific for the target molecule associated with the oligomeric metal coordination complex; and
(b) contacting the binding support with the sample comprising the target molecule, to thereby capture the target molecule from the sample.

In a fourth aspect of the disclosure, there is provided an assay comprising:
(a) contacting a binding support with a target molecule, the binding support comprising:
   (i) a polymeric porous substrate formed from at least one polymer;
   (ii) an oligomeric metal coordination complex associated with the at least one polymer forming the polymeric porous substrate;
   (iii) at least one affinity agent specific for the target molecule associated with the oligomeric metal coordination complex; and
(b) detecting the binding of the target molecule with the target-specific affinity agent.

In embodiments, the affinity assay is an immunoassay.

In certain embodiments, the assay is a lateral flow assay.

In embodiments of the first to fourth aspects, the oligomeric metal coordination complex forms an affinity network comprising a network of metal coordination complexes coordinately bonded to a plurality of affinity agents.

In embodiments of the first to fourth aspects, the metal coordination complex forms an affinity network of a plurality of metal coordination complexes coordinately bonded to and interconnecting a plurality of affinity agents.

In embodiments of the first to fourth aspects, the plurality of affinity agents may be a plurality of biomolecules specific for one or more target molecules.

The various features and embodiments of the present disclosure, referred to in individual sections above apply, as appropriate, to other sections, mutatis mutandis. Consequently, features specified in one section may be combined with features specified in other sections as appropriate.

### BRIEF DESCRIPTION OF THE DRAWINGS

FIG. 1 shows the zeta size of Bovine Serum Albumin (BSA) aggregates formed when unmodified oligomeric metal complexes (Solution 1) is added after (a) 1 minute, (b) 30 minutes.

FIG. 2 shows the zeta size of Bovine Serum Albumin (BSA) clusters after the addition of different concentrations modified oligomeric metal complexes, (a) Solution 3A and (b) Solution 3B over 7 hours.

FIG. 3 shows the zeta size of Bovine Serum Albumin (BSA) clusters formed when modified oligomeric metal complexes (Solution 4) is added at room temperature after (a) 1 minute, (b) 10 minutes, (c) 20 minutes and (d) 60 minutes.

FIG. 4 shows the zeta size of Bovine Serum Albumin (BSA) clusters formed when modified oligomeric metal complexes (Solution 4) is added at 37° C. after (a) 1 minute, (b) 10 minutes, (c) 60 minutes, and (d) 120 minutes.

FIG. 5 shows the zeta size of the Bovine Serum Albumin (BSA) clusters formed using different concentrations of modified oligomeric metal complexes, (a) Solution 4, (b) Solution 5A and (c) Solution 5B over 7 hours.

FIG. 6 shows the signal intensity in RFU of the capture line of a lateral flow test strip on which mouse IgG antibody conjugated to a nanoparticle (detection moiety) is captured by the goat anti-mouse antibody, passively immobilised, at three different pH values (pH 5.2, 6.0 and 8.5) to nitrocellulose membranes. The fluorescence intensity is an indicator of the number of Europium nanoparticles (detection moiety) captured on the capture line.

FIG. 7 shows the signal intensity in RFU of the capture line of a test strip where the mouse IgG antibody conjugated to a nanoparticle (detection moiety) is captured by the goat anti-mouse antibody cross-linked by an oligomeric metal complex (Solution 4) at two concentrations (0.1 mM and 0.5 mM) and at three different pH values (pH 5.2, 6.0 and 8.5) striped onto nitrocellulose membrane. The fluorescence intensity is an indicator of the number of Europium nanoparticles captured on the capture line. For comparison purposes, the signal obtained via passive binding at pH 8.5 (Control) is shown on the far right.

FIG. 8 shows the signal intensity in RFU of the capture line where the mouse IgG antibody Conjugate is captured by the cluster formed with goat anti-mouse antibody and an oligomeric metal complex (Solution 4) mixed and left for different times before striping onto nitrocellulose membranes.

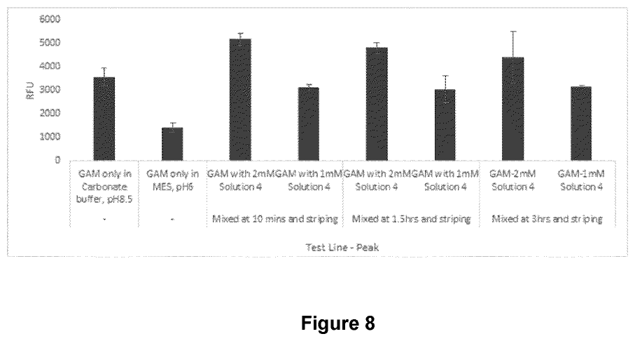

FIG. 9 shows the signal intensity in RFU of the capture line where the mouse IgG antibody Conjugate is captured by the cluster formed with goat anti-mouse antibody and an oligomeric metal complex (Solution 4) on the nitrocellulose membrane. The membrane was pre-blocked with BSA prior to striping of antibody clusters.

FIG. 10 shows the signal intensity in RFU of the capture line where the mouse IgG antibody Conjugate is captured by the cluster formed with goat anti-mouse antibody and an oligomeric metal complex (Solution 6C) in carbonate buffer (pH 8.5).

FIG. 11 shows the relative signal intensity (Sn/S0) of FluA antigen in a lateral flow sandwich assay with capture by clusters formed with anti-FluA antibody and an oligomeric metal complex (Solution 6C).

Further aspects of the present invention and further embodiments of the aspects described in the preceding paragraphs will become apparent from the following description, given by way of example and with reference to the accompanying drawings.

### DETAILED DESCRIPTION OF THE EMBODIMENTS

The present invention is predicated, at least in part, on the understanding that certain oligomeric metal coordination complexes can be used to cross-link biologically active molecules, such as affinity agents including antibodies or antigens, in a controlled manner such that their functionality and specificity of binding are retained. The cross-linking can be achieved while avoiding forming tightly bound aggregates of the affinity agents within metal coordination complex matrices which might not be functionally available for their capture role. Rather, the level of control allows for the cross-linked metal coordination complexes with bound affinity agent to form at some controlled rate and cluster size even after application to a polymeric porous substrate. This provides in one step, for the forming network of metal coordination complexes, with bound affinity agent, to become localised at some pre-determined regions of a polymeric porous substrate via oligomeric metal complex and/or via passive binding. It may also provide for the forming network of metal coordination complexes, with bound affinity agent, to become at least partially interpenetrating with the polymeric porous substrate rather than simply adhering to the porous substrate surface through passive binding. This approach has been found to provide for some further benefits in improving the sensitivity of detection of a target molecule binding event to the affinity agent.

The disclosure herein provides for a straightforward and reliable approach to effectively convert an affinity agent, or a combination of different affinity agents, into a polymeric or interconnected form. It is believe that the polymeric or interconnected form may at least partially interpenetrate or interlace, on a molecular scale, with a polymeric porous substrate material. In the case of membranes used in lateral flow assays, this approach may have a number of further benefits. For example, it may help minimise functional damage due to passive binding. In passive binding, the dominant interactions are dipole-dipole, ionic, hydrogen bonding, hydrophobic and other non-covalent interactions and, as a consequence, the outcome can vary considerably with the nature of the affinity agent and the immobilisation conditions, such as the buffer employed and the pH. Passive immobilisation approaches can lead to protein damage, poor orientation and stacking of proteins leading to steric crowding. For another example, formation of a polymeric or interconnected form of affinity agents may increase overall mobility and accessibility enhancing its binding efficiency to target molecule. The benefits of the present approach may include, but are not limited to, an essentially one-step immobilisation process, the lack of any complicated chemical modifications, minimising non-specific background signal, improved binding functionality and/or assay sensitivity, and that the process is controllable to assemble and screen various affinity agent constructs to identify more mobile and accessible affinity agent complexes to provide optimal results for any given assay.

The use of surface immobilised binding with unmodified metal complexes, such as those described in Conjugating Molecules to Particles (PCT/AU2014/050181) in the name of the present applicant, can provide for protein binding which is both extremely rapid and strong. The benefits of high reactivity do, however, raise a significant challenge in that if the metal complexes were not first immobilised on a surface of a particle, it does not form the intended product. Instead, an unusable mixture comprising of aggregated particles, uncontrolled cross-linked capture agents and their combinations are formed due to the high and uncontrolled reactivity of the metal complexes. The metal complexes and conditions used in this application have significantly slower reactivity allowing the formation of stable and uniform affinity agent clusters via coordination interactions in the presence of the porous substrate, which may at least partially interpenetrate the porous substrate and localize the polymeric affinity agent-metal complex to a specific location on the porous substrate.

### Definitions

As used herein, the term “interpenetrating” or “interpenetrating polymer network (IPN)” describes a blend of two or more polymers in a network with at least one of the polymer systems synthesised or formed, at least in part, in the presence of the other. This results in the formation of a physically cross-linked network in which polymer chains of the second system are entangled with or penetrate the network formed by the first polymer. Particularly, the oligomeric metal coordination complexes described herein may be mixed with the at least one affinity agent and the cross-linked interconnected affinity network they form with that agent may be considered polymeric and this is applied, during its formation, to a polymeric porous substrate while the cross-linking is still occurring, such that the interpenetration or interlacing of the forming affinity agent-metal complex polymer into the polymeric porous substrate occurs, at least to some extent, at the molecular level to form a IPN or IPN-like arrangement.

As used herein, the term “semi-interpenetrating polymer network (SIPN)” may be considered to describe a polymer comprising one or more polymer networks and one or more polymers characterised by the penetration on a molecular scale of at least one of the networks by at least some of the polymer macromolecules. A SIPN may be distinguished from an IPN because the constituent polymer macromolecules can, in principle, be separated from the constituent polymer network without breaking chemical bonds; i.e. it is a polymer blend. In embodiments, the forming interconnected affinity network described above may form a SIPN with the polymer(s) of the polymeric porous substrate.

As used herein, the term “affinity agent” or “at least one affinity agent” is broad enough to include within its scope both capture agents, such as antibodies as may be used in a sandwich assay, and analytes, as may be used in a competitive assay, which are associated with the oligomeric metal coordination complexes described herein. So long as the agent associated with the oligomeric metal coordination complexes can participate in a binding event having appropriate specificity with a target molecule (which equally may be an antibody or analyte) in a sample of interest then it may, for the purposes of the present disclosure, be considered an affinity agent. In embodiments, the “affinity agent” or “at least one affinity agent” may be a “capture agent” or “at least one capture agent”, being a larger biomolecule which has a specific binding affinity for a smaller analyte of interest.

As used herein the term “affinity agent cluster” may include at least two or more affinity agents bound by oligomeric metal coordination complexes to form a plurality or multiplicity of such binding agents in the form of an interconnected affinity network cluster. The process includes an expansion in the size of the affinity network cluster and depending on the timing of the inclusion of the porous substrate, intermediate clusters can be further expanded by the inclusion of more of the same affinity agent or a different affinity agent or some protein or synthetic polymer acting as an spacer for the affinity agent.

As used herein the terms “specificity” (as it relates to binding), “specific binding”, “specifically binds”, and the like, indicates that the affinity agent binds preferentially to the target molecule or analyte of interest or binds with greater affinity to the target (analyte) than to other molecules within a sample. For example, an antibody will selectively bind to the antigen against which it was raised. A DNA molecule will bind to a substantially complementary sequence and not to unrelated sequences under stringent conditions. Specific binding can refer to a binding reaction that is determinative of the presence of a target in a heterogeneous population of molecules (e.g., proteins and other biologics). Thus, under designated conditions (e.g. immunoassay conditions in the case of an antibody or stringent hybridisation conditions in the case of a nucleic acid), the specific ligand or antibody binds to its particular “target” molecule and does not bind in a significant amount to other molecules present in the sample.

In a second aspect of the disclosure, although not necessarily the broadest aspect, there is provided a binding support comprising:
(a) a polymeric porous substrate formed from at least one polymer;
(b) an oligomeric metal coordination complex associated with the at least one polymer forming the polymeric porous substrate; and
(c) at least one affinity agent associated with the oligomeric metal coordination complex.

The binding support may be a component of any device which requires a porous substrate presenting an affinity agent to be exposed to a liquid sample to achieve a binding event with appropriate specificity.

The binding support may be a component of a filtration system, a chromatographic system, a controlled drug release approach, a medical device for implantation or any other such application. The term IPN, SIPN and IPN-like arrangements have been used to describe different constructs ranging from hydrogels to microporous structures for many different applications. The porous substrate describes any support with porosity (micro- to macroporous) which may allow for some partial interpenetration of affinity agent and metal complex.

In embodiments, the binding support may be a component of an assay device.

In embodiments, the binding support may be a component through which a fluid sample flows within an assay device.

In embodiments, the binding support may be, or may be a part of, a test strip in an assay device.

It will be appreciated that it is not required, or even necessarily preferable, that the binding support has the metal coordination complexes, with associated at least one affinity agent, throughout the extent of the polymeric porous substrate. The extent of this arrangement will also depend upon the purpose for which the binding support is to be used. If, for example, the binding support is a component of a chromatography system then the metal coordination complex-affinity agent network (the interconnected affinity network) may be associated with a substantial portion or all of the porous substrate which is to be exposed to the sample containing the target molecule of interest. On the other hand, if the binding support is, for example, a test strip for a lateral flow assay then the metal coordination complex-affinity agent network may only be striped onto a specific region of the polymeric porous substrate corresponding to the test line and/or control line. To localize the metal coordination complex-affinity agent to a specific region, such as striping a capture line on a lateral flow membrane, using prior art approaches cannot lead to comparable outcomes to those presented herein. Striping a membrane firstly with a standard metal complex (such as Solution 1 in the Examples) and then attempting to stripe exactly the same location with affinity agent is technically challenging and any minor error could lead to binding of target molecule thus invalidating the whole assay. The highly reactive metal complexes used in the prior art can coordinate with the membrane resulting in blockages within the pores and, at best, any two-step process will lead to surface immobilised binding with unmodified metal complexes such as are described in the prior art. They do not provide for the formation of metal coordination complex-affinity agent networks formed in the presence of the binding support.

The nature of the polymeric porous substrate is not particularly limited so long as it allows for the flow of a liquid sample at an appropriate rate. The polymer or polymers forming the polymeric porous substrate may be natural or synthetic. Synthetic polymers may be wholly synthetic or modified or derivatised natural polymers. The nature of the polymer will affect the degree to which an affinity agent network interpenetrates the at least one polymer forming the polymeric porous substrate as many polymers has a natural tendency to passively bind proteins and/or be able form coordination interactions with metal complexes.

Depending on the application, the porosity, pore size and thickness of the porous substrate may be selected based on parameters which are well-known in the art. For example, if the binding support is for a lateral flow test then the polymeric porous substrate may be nitrocellulose and/or other material components of the flow test including sample and conjugate pads. The specific binding support selected will be one with a desired pore size, pore volume and thickness as these parameters all impact on the speed of travel of the fluid sample along the binding or test strip and the amount of sample which can be utilised. Nitrocellulose is traditionally used as it allows easy striping of capture and control lines and, in standard approaches, the affinity agent is binding via physical/passive absorption. For the purposes of the present disclosure, it is not necessary for the porous substrate to be non-reactive to physical/passive absorption as long as there are metal coordination complex-affinity agent networks being formed in the presence of the porous substrate. Depending on the substrate material, the network may include physical/passive absorption and/or partial interpenetration of porous substrate.

In embodiments, the polymeric porous substrate may comprise one or more polymers or materials selected from the group consisting of nitrocellulose, cellulose, glass fibre, polymers of 2-hydroxyethyl methacrylate (HEMA), 2-hydroxypropyl methacrylate (HPMA), acrylamide (AAm), N-isopropylacrylamide (NIPAm), and methoxyl poly (ethylene glycol) (PEG) monoacrylate (mPEGMA or PEGMA), with cross-linkers, such as N,N′-methylenebis(acrylamide) (MBA), ethylene glycol diacrylate (EGDA) and PEG diacrylate (PEGDA). Poly (N-isopropylacrylamide) (PNIPAm), one or more naturally-occurring polymers (e.g., cellulose or hyaluronan-based hydrogels), while in other embodiments, the hydrogels can comprise one or more synthetic polymers (e.g. polyethylene glycol dimethacrylate, urethane dimethacrylate, etc.).

If the polymeric porous substrate comprises a hydrogel then illustrative hydrogels include, but are not limited to polylethylene glycol hydrogels, polyethylene oxide hydrogels, polyphosphazene hydrogels, collagen hydrogels, polysaccharide hydrogels, hydroxyethyl methacrylate hydrogels, acrylic hydrogels, copolymers of polyoxyethylene/polyoxypropylene/polyoxyethylene hydrogels, alginate hydrogels, gelatin based hydrogels, chitosan based hydrogels, dextran-aldehyde conjugate hydrogels, hyaluronan/gelatin hydrogels, acrylamide/itaconic acid copolymer hydrogels, acrylic hydrogels, nanometal hydroxide hydrogels, poly (N-vinyl pyrrolidone) hydrogels, poly (N-isopropylacrylamide) hydrogels, collagen-chondroitin sulfate hyaluronic acid hydrogels, polyacrylic acid hydrogels, polyvinyl alcohol hydrogels, and the like.

In embodiments, the porous substrate may be a nitrocellulosic or cellulosic porous substrate. Such a substrate may, in embodiments, form all or part of a test strip for a lateral flow assay (LFA), otherwise referred to as a lateral flow test (LFT).

In embodiments, the metal ion of the oligomeric metal coordination complex is selected from the group consisting of chromium, ruthenium, iron, cobalt, titanium, aluminium, zirconium, and combinations thereof. In embodiments, the metal ion of the metal coordination complex is selected from the group consisting of chromium, ruthenium, titanium, iron, cobalt, aluminium, zirconium, rhodium and combinations thereof.

In preferred embodiments, the metal ion is chromium.

The metal ion of the oligomeric metal coordination complex may be present in any applicable oxidation state. For example, the metal ion may have an oxidation state selected from the group consisting of I, II, III, IV, V, or VI, as appropriate and obtainable under standard conditions for each individual metal. The person of skill in the art would be aware of which oxidation states are appropriate for each available metal.

In an embodiment in which the metal ion is a chromium ion, it is preferred that the chromium has an oxidation state of III.

The metal ion may be associated with any suitable counter-ions such as are well-known in metal-ligand coordination chemistry.

In certain embodiments, mixtures of different metal ions may be used, for example, to form a plurality of different oligomeric metal coordination complexes. In such cases, it is preferred that at least one metal ion is chromium.

Metals are known to form a range of oligomeric metal coordination complexes. Preferred ligands for forming the oligomeric metal coordination complex are those that include nitrogen, oxygen, or sulfur as dative bond forming groups. More preferably, the dative bond forming groups are oxygen or nitrogen. Even more preferably, the dative bond forming group is an oxygen-containing group which assist in olation to form the oligomeric complexes. In embodiments, the oxygen-containing group is selected from the group consisting of oxides, hydroxides, water, sulphates, phosphates, or carboxylates.

In embodiments, the oligomeric metal coordination complex is a chromium (III) oligomeric metal coordination complex. In embodiments, the oligomeric metal coordination complex is an oxo-bridged chromium (III) oligomeric coordination complex. This complex may optionally be further oligomerised with one or more bridging couplings such as carboxylic acids, sulphates, phosphates and other multi-dentate ligands.

Exemplary oxo-bridged chromium structures as described in the applicant's prior filings are provided below:

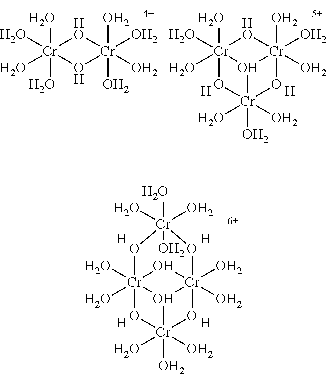

On contact with some substrate, such as a particle as described in Conjugating Molecules to Particles (PCT/AU2014/050181), at least one of the water or hydroxyl groups (or whatever ligands may be present) on the oligomeric metal coordination complexes is replaced by a dative bond with the substrate surface. This is illustrated below wherein “X” represents the dative bond to the substrate surface.

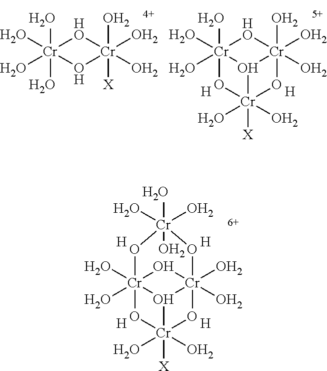

It will also be appreciated that multiple water or hydroxyl or other ligands present on the oligomeric metal coordination complex may be replaced by a dative bond with the substrate surface but when such oligomeric metal complexes are added in excess, and then the excess washed off the substrate, what remains is an oligomeric metal complex activated substrate which has several useful properties. Due to its size and oligomeric nature, on binding to a substrate there still remains coordination potential to bind affinity agents or other polymers or materials. The binding to affinity agents and the like is highly reactive due to the multiplicity of coordination sites which gives multi-component or avidity binding characteristics driven by multiple charge—charge and coordination interactions. As previously described, it is critical to first add such oligomeric metal complexes in excess to some substrate, and then wash off the excess to form an oligomeric metal complex activated substrate.

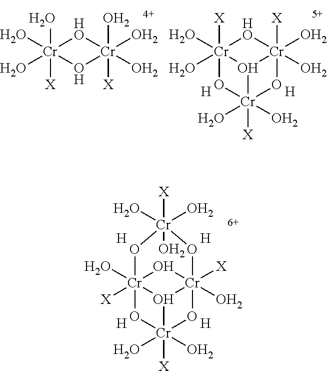

Given the above discussion, any mixing of such an oligomeric metal complex with an affinity agent will lead to a rapid, uncontrolled reaction resulting in insoluble affinity agent aggregates in a matter of seconds. It will be appreciated that one possible outcome of the present disclosure in terms of an interpenetrating network within the polymeric porous substrate cannot be achieved with such unmodified oligomeric metal complexes.

However, if the oligomeric metal coordination complexes to which the at least one affinity agent is exposed are as set above but additionally are modified in terms of a ‘tuning down’ of their reactivity, then uncontrolled oligomeric metal complex-affinity agent aggregates need not form. Again without wishing to be bound by theory, it is believed this means that when a liquid formulation of the so modified oligomeric metal coordination complexes and at least one affinity agent is applied to a polymeric porous substrate shortly after preparation of the liquid formulation, the forming interactions between the oligomeric metal coordination complexes and at least one affinity agent occur over a desirable timeframe in the presence of the polymeric matrix of the porous substrate and therefore the network formed may interpenetrate or interlace that matrix, at least partially. This is as opposed to ‘standard’ or ‘unmodified’ oligomeric complexes which, as previously described, are highly reactive and would simply react completely almost immediately after contact with the at least one affinity agent to form insoluble and non-functional affinity agent aggregates.

The solution presented herein is therefore to employ modified oligomeric metal coordination complexes, being relatively unreactive metal complexes, which will form bonds to the affinity agents at an appropriate rate to allow sufficient time for the liquid formulation to be applied to the polymeric porous substrate prior to final formation of the interconnected affinity network. Without wishing to be bound by theory, the inventors believe that by controlling the kinetics of coordination of the modified oligomeric metal coordination complex to the affinity agent, it may be possible to form a stable interconnected affinity network within and interpenetrating the polymer(s) of the porous substrate.

The degree of modification of any particular oligomeric metal coordination complex can be achieved, for example the extent or excess of smaller coordinating agents (described as capping groups) to form the modified oligomeric metal coordination complex. As coordination interactions are reversible, a larger molecule such as an affinity agent having multiple coordination potential will over time, compete off a small coordinating agent. The stability of such coordinating agents to exchange is dependent on the pH, the basic coordination strength, multi-valency, method of synthesis and as well any extra coordinating agents (same or different) are present is the liquid formulation. The use of such modifications can delay coordination of such modified oligomeric metal complexes to affinity agent from minutes, to hours or even days.

In embodiments, the modified oligomeric metal coordination complex may be defined as a reduced reactivity oligomeric metal coordination complex, especially relative to the same oligomeric metal complex which is fully hydrated (for example a complex formed with non-coordinating counter-ions), such as described as Solution 1 in the Examples.

In embodiments, the modified oligomeric metal coordination complex is modified such that its reactivity is reduced as compared with the same oligomeric metal coordination complex which has not been so modified, for example the same metal coordination complex but with non-coordinating counter-ions in a fully hydrated state (for example in the form of a hexahydrate). In one embodiment, the unmodified metal coordination complex has non- or weakly coordination anions as ligands.

In embodiments, the reduced reactivity of the modified oligomeric metal coordination complex may be defined as a reduced level of reactivity as compared with an otherwise corresponding unmodified metal complex, for example an unmodified oxo-bridged chromium (III) complex. The unmodified metal complex may be a fully hydrated metal complex. The oxo-bridged chromium (III) complex may be a fully hydrated oxo-bridged chromium (III) complex.

In embodiments, the unmodified oxo-bridged chromium (III) complex used for comparison purposes may be that as formed in ‘Solution 1’ of Example 1 in the examples section.

In embodiments, the modified oligomeric metal coordination complex is modified such that its reactivity to, or speed to bond with, the at least one affinity agent is reduced as compared with the same oligomeric metal coordination complex which has not been so modified. Without wishing to be bound by theory, it is believed this means that when a liquid formulation of the modified oligomeric metal coordination complex and the affinity agents in solution is generated, then the multiplicity of potential coordinating ligands on the affinity agents will have changed. Unlike synthetic polymers, which have repeating ligands with the same metal coordination potential, biological polymers, such as proteins, include different types of ligands with different strengths of coordination to metal complexes. The addition of capping agents sets a threshold by which only a limited number of ligands presenting on the affinity agent can coordinate. For example, uncontrolled coordination of metal complexes to any ligand in the affinity agent is minimised, as only a limited number of ligands can coordinate per individual affinity agent, which encourages intermolecular cross-linking of affinity agents by the oligomeric metal complexes. At the extreme, too much capping or competing agents for a set amount of oligomeric metal complex will prevent formation of an affinity agent cluster while the opposite will lead to multiple intra-molecular coordination leading to conformational damage of the affinity agents by the oligomeric metal complex. The ability to adjust the conditions prevents a non-functional tightly bound aggregate from forming. The control of reactivity may also allow time for uniform mixing and thus the opportunity for forming at least partially interpenetrating affinity agent networks in the presence of porous substrates.

In embodiments, the time by which 50% of the available coordination capacity of the modified metal coordination complex is taken up by the affinity agent network is greater than the time by which 50% of the available coordination capacity of the directly corresponding but unmodified metal coordination complex is taken up by the same affinity agent applied at the same relative amounts and/or concentrations.

As the modified oligomeric metal coordination complexes form bonds with the at least one affinity agent and, to some extent, the porous substrate (depending on the substrate) and each other, they form an interconnect affinity network and would, following this bonding process, no longer be considered to be ‘modified’, particularly following drying and/or curing.

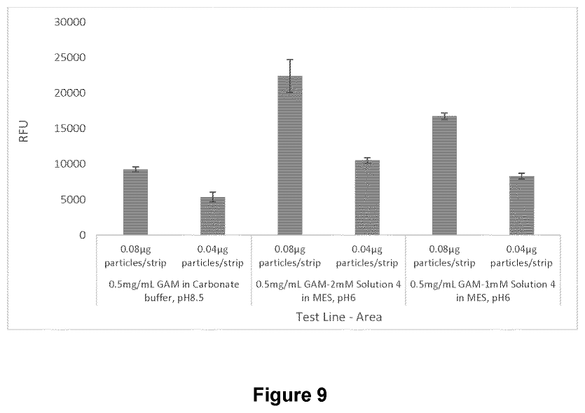

It will be appreciated again that this is a dynamic system and so when the liquid formulation of oligomeric metal coordination complexes and at least one affinity agent are applied to the polymeric porous substrate, as described above, there will be some at least one affinity agent which becomes passively bound (in the case of such substrates) as well as some oligomeric metal coordination complexes which bind to the porous substrate surface and then further oligomeric metal coordination complexes with associated at least one affinity agent may bind to these. Without wishing to be bound by theory, it is believed at least a portion of the oligomeric metal coordination complexes and associated at least one affinity agent may interpenetrate or interlace the polymeric porous substrate at the molecular level to thereby provide some further benefits of the present disclosure. As is demonstrated in the examples, it has been shown that depending on the affinity agent and modified oligomeric metal complex ratios, the type of modification and its excess, changes the characteristics of the affinity agent clusters to bind target molecules. For example, the benefits of the present disclosure can be observed even when a porous substrate has been passivated with a passivating agent and so the arrangement provided is one with significant contributions which cannot be said to be a result of passive binding (Example 6, FIG. 9).

As discussed above, the oligomeric metal coordination complex and associated affinity agent are associated with and/or may also be at least partially interpenetrating the at least one polymer forming the polymeric porous substrate. Depending on the substrate, the association may include a physical association such as an interpenetrating relationship and/or a bonding association based on coordination bonding between the oligomeric metal coordination complex and any electron-donation groups of the at least one polymer forming the polymeric porous substrate. It is believed that in most instances there may be a physical association due to the interpenetrating or semi-interpenetrating relationship and/or coordination bonding which will be varied in nature, in that it may include:
(i) oligomeric metal coordination complex with bonded affinity agent directly coordinately bonded to the at least one polymer of the porous substrate; and/or
(ii) oligomeric metal coordination complex without bonded affinity agent but to which further oligomeric metal coordination complexes having affinity agent bonded thereto will become associated with.

A person of skill in the art will appreciate this and a number of other scenarios are possible at any point in time based on the type and dynamic nature of the components.

While the discussion in relation to this second aspect of the disclosure relates to an oligomeric metal coordination complex associated with and/or at least partially interpenetrating the polymeric porous substrate, and also associated with at least one affinity agent, it will be appreciated that the oligomeric metal coordination complexes may no longer exist as discrete oligomeric complexes or may not be able to be truly identified as such once the product has formed and cured. This is because the association with the at least one affinity agent will result in multiple previously separate oligomeric metal coordination complexes being bound to each affinity agent. In solution, some of these individual coordinate bonds will break and reform with the same or different affinity agents and so what might be termed an affinity or capture network, or interconnected affinity or capture network, of interconnected affinity agents is formed with the formerly oligomeric metal coordination complexes essentially forming the strands or connections of the network between separate affinity agents as hubs and with at least a portion of the metal coordination complex connections associated with the polymeric porous substrate at the molecular level. Based upon the interconnected network formed, the affinity or capture network may be viewed as polymeric in nature even though it has been formed, in part, from oligomeric metal coordination complexes. For this reason the affinity or capture network may be viewed as forming a IPN or SIPN with the at least one polymer forming the polymeric porous substrate.

In embodiments, the oligomeric metal coordination complex may form an interconnected affinity network of a plurality of metal coordination complexes coordinately bonded to a plurality of affinity agents and at least a portion of the metal coordination complexes of the interconnected affinity network may be at least partially interpenetrating the at least one polymer forming the polymeric porous substrate. At least partially interpenetrating the at least one polymer forming the polymeric porous substrate may, in some embodiments, mean forming a SIPN and/or a IPN or IPN-like arrangement with said polymeric porous substrate.

In some embodiments, the oligomeric metal complex may form an interconnected affinity network including additional proteins that are not affinity agents and/or synthetic polymers. In such embodiments, the additional proteins may be employed simply to separate or space out the affinity agents to a desirable degree and so such proteins, in the context of the interconnected affinity network, may be considered non-functional. In other embodiments, such additional proteins may serve a related but non-identical function to the affinity agents. Synthetic polymers may also be introduced with the forming interconnected affinity network to serve a similar spacing purpose within the network. The use of such additional proteins will be dependent on the nature of the application and/or the nature of the affinity agents being used.

It will be appreciated that the oligomeric metal coordination complex described herein can provide for the formation of interconnected affinity network clusters or polymeric interconnected affinity network clusters in situ where the oligomeric metal coordination complex can form affinity agents clusters as well as binding to polymeric porous substrate.

In some embodiments, the interconnected affinity network may be at least partially interpenetrating the at least one polymer forming the polymeric porous substrate.

In some embodiments, the polymeric interconnected affinity network may be at least partially interpenetrating the at least one polymer forming the polymeric porous substrate.

Without wishing to be bound by theory, it is believed that the forming interconnected affinity network or forming interconnected polymeric affinity network may result in the individual affinity agents also becoming interconnected with and dispersed throughout the polymeric porous substrate rather than just the strands of metal coordination complex connecting the affinity agents. Viewed on a size basis it may be considered that this is more so in terms of affinity agent being throughout the pore of the porous substrate as typical affinity agents, such as antibodies, are considerably larger than individual oligomeric metal coordination complexes. When viewed as an entire interconnected affinity network it can therefore be understood that, in some embodiments, a significant proportion, such as more than half or such as the majority of the mass or surface area of the interconnected affinity network which is within the polymeric porous substrate is represented by the affinity agent component of said network.

Therefore, in some embodiments, there may be provided a binding support comprising:
(a) a polymeric porous substrate formed from at least one polymer;
(b) an affinity network comprising a plurality of affinity agents associated with a metal coordination complex; and
(c) the affinity network at least partially interpenetrating the at least one polymer forming the polymeric porous substrate.

In embodiments, the affinity network may be an interconnected affinity network.

In embodiments, the affinity network may be a polymeric affinity network comprising the plurality of affinity agents associated with and connected by metal coordination complexes.

In embodiments, the affinity network may be a polymeric interconnected affinity network comprising the plurality of affinity agents coordinately bonded with, and interconnected by, metal coordination complexes.

In embodiments, the at least one, or plurality of, affinity agents may be a biomolecule specific for a target molecule. That is, the affinity agent biomolecule and the target molecule may be binding partners.

In embodiments, the at least one, or plurality of, affinity agents may be an at least one capture agent.

In embodiments, the at least one, or plurality of, affinity agents may be an at least one antigen agent.

In embodiments, the at least one, or plurality of, affinity agents may be a protein and/or nucleic acid-based affinity agent.

In certain embodiments the at least one, or plurality of, affinity agents may be independently selected from the group consisting of an antibody, an antigen, a monoclonal antibody, a polyclonal antibody, an antibody fragment, an antibody peptide, an antibody mimetic, an antibody fusion protein, a phage display, a nucleic acid aptamer, a fibronectin display, a peptide-nucleic acid aptamer, and a non-antibody protein scaffold.

In some embodiments, the affinity agents may be independently selected from antigen binding proteins, such as polyclonal antibodies, monoclonal antibodies and antigen binding fragments thereof, that bind specifically to one or more of: SARS-COV-2, human immunodeficiency virus (HIV), hepatitis, malaria, respiratory syncytial virus (RSV), Ebola virus (EBOV), human cytomegalovirus (HCMV) and influenza. For example, the affinity agents may be independently selected from antigen binding proteins, such as antibodies and antigen binding fragments thereof, that specifically bind to CoV spike or nucleocapsid protein, influenza hemagglutinin or nucleocapsid, or an antigen fragment thereof.

It will be appreciated that the polymer-like entity formed by cross-linking of the at least one affinity agent and the oligomeric metal coordination complexes is a dynamic system due to the nature of the association between the two components. Without wishing to be bound by theory, it is postulated that the oligomeric metal coordination complexes are associated with the at least one affinity agent through avidity or multi-component bonding and so the at least one affinity agent is directly bonded to the oligomeric metal coordination complex through multiple coordinate bond interactions the accumulated strength of which results in anchoring of the at least one affinity agent to the oligomeric metal coordination complex as if it were bonded via standard covalent bonding. As discussed, this may be viewed as forming an interconnected affinity network or polymeric interconnected affinity network. However, any individual coordination bond between the metal ion in the oligomeric metal coordination complexes and the at least one affinity agent is relatively weak and can break as a result of a local stressor which allows for freedom of movement or orientation allowing the affinity agent to be optimally functionally available.

It will be appreciated that there may be at least about 30% more inter-molecular binding of affinity agents with the oligomeric metal coordination complex described herein to form interconnected affinity network clusters or polymeric interconnected affinity network clusters of controllable size and enhanced binding to its binding partner or target molecule than the same oligomeric metal coordination complex which has not been so modified. In some embodiments, there may be at least about 30%, 40%, 50%, 60%, 70%, 80%, 90%, or 95% more inter-molecular binding of affinity agents with the oligomeric metal coordination complex described herein to form interconnected affinity network clusters or polymeric interconnected affinity network clusters than the same oligomeric metal coordination complex which has not been so modified.

In embodiments of the second aspect of the disclosure, there is provided a binding support comprising:
(a) a polymeric porous substrate formed from at least one polymer; and
(b) an interconnected affinity network formed from metal coordination complexes associated through coordinate bonds with, and interconnecting, a plurality of affinity agents, the interconnected affinity network associated with the at least one polymer forming the polymeric porous substrate in a defined region of said porous substrate to form a binding strip.

In some embodiments of the second aspect of the disclosure, there is provided a binding support comprising:
(a) a polymeric porous substrate formed from at least one polymer; and
(c) an interconnected affinity network formed from metal coordination complexes associated through coordinate bonds with, and interconnecting, a plurality of affinity agents, the interconnected affinity network at least partially interpenetrating the at least one polymer forming the polymeric porous substrate in a defined region of said porous substrate to form a binding strip.

The polymeric porous substrate, oligomeric metal coordination complexes, and affinity agents may all be selected from those previously described for the second aspect.

In embodiments, the binding strip may be a test line and the binding support may be a test strip of a LFA.

The components of a LFA are well-known in the art but, briefly, a LFA comprises a sample pad, a conjugate pad, a nitrocellulose support or strip that contains test and control lines, and a wicking pad. Each component will typically overlap to encourage appropriate capillary flow of the test sample. To use the device, a liquid sample such as blood, serum, plasma, urine, saliva, or solubilised solids, is added directly to the sample pad and from there is wicked through the lateral flow device. The sample pad neutralises the sample and filters unwanted particulates allowing the sample to flow unimpeded to the conjugate pad which contains, for example, strongly coloured or fluorescent nanoparticles that are provided with an antibody on their surface. In one embodiment, the nanoparticles may be colloidal gold as is common in commercial LFAs. When the liquid reaches the conjugate pad, these dried nanoparticles are released and mix with the sample. If there are any target analytes in the sample that the antibody recognises, these will bind to the antibody. The analyte-bound nanoparticles then flow through the nitrocellulose membrane and across one or more test lines and a control line. The test line is the primary read-out of the diagnostic and consists of immobilised affinity agents that can bind the nanoparticle to generate a signal that is correlated to the presence of the target molecule/analyte in the sample. The fluid continues to flow across the strip until it reaches the control line. The control line contains affinity ligands that will bind the nanoparticle conjugate with or without the target molecule/analyte present in solution to confirm that the assay is working properly. After the control line, the fluid flows into the wicking pad which is needed to absorb all of the sample liquid to ensure that there is consistent flow across the test and control lines. Once all the sample has passed across the test and control lines, the assay is complete and the user can read the results.

It will be appreciated that, in embodiments, the binding support of the second aspect may be a component of a lateral flow device. Particularly, the binding support may be or form a part of the test strip of the lateral flow device. The region of binding support that may have the interconnected affinity network formed thereon may be the test and/or control lines of the test strip.

In a first aspect of the disclosure, there is provided a method of forming a binding support, the binding support comprising a polymeric porous substrate formed from at least one polymer; an oligomeric metal coordination complex associated with the at least one polymer forming the polymeric porous substrate; and at least one affinity agent associated with the oligomeric metal coordination complex, the method including the steps of:
(a) providing a polymeric porous substrate formed from at least one polymer;
(b) providing a liquid formulation comprising an oligomeric metal coordination complex associated with at least one affinity agent, within a liquid carrier; and
(c) contacting the polymeric porous substrate with the liquid formulation, to thereby form the binding support.

The binding support, polymeric porous substrate, oligomeric metal coordination complex and at least one affinity agent may be as described for any embodiment, or combination of embodiments, of the second aspect.

As discussed, the advantages of the present disclosure may relate, at least in part, to the ability to have the metal coordination complexes linking the affinity agent also be at least partially interpenetrating the at least one polymer forming the polymeric porous substrate. This approach is demonstrated within the examples to provide for improvements in functional availability and/or mobility of the affinity agent as demonstrated by improved signal and/or decrease background versus simple passive immobilisation.

In forming such an interconnected affinity network it is therefore key to ensure that at the time the liquid formulation comprising the oligomeric metal coordination complexes associated with the at least one affinity agent is contacted with the polymeric porous substrate that the oligomeric metal coordination complexes have not fully bonded with the affinity agent. If this occurs then the oligomeric metal coordination complexes cannot interpenetrate the porous substrate at the molecular level. Indeed, the applicant has found that simply forming such clusters of oligomeric metal coordination complexes full coordinately bonded to the affinity agents and then passively immobilising these onto a nitrocellulose test strip provides for an outcome which is not significantly different to simply immobilising the affinity agent alone onto the test strip. The advantages are only seen when the liquid formulation can be applied while the oligomeric metal coordination complexes are still forming bonds with the affinity agent to generate the interconnected affinity network.

This is in contrast to Method of Controlled Competitive Exchange (PCT/AU2016/051132), also in the name of the present applicant, which describes a first bound agent immobilised on a substrate via a metal complex solely for the purpose of exchange by a competing agent, i.e. competitive exchange. In the embodiment described in Examples 3 and 4 of Method of Controlled Competitive Exchange, the first agent capped metal complex is added in excess to the membrane so that there is residual coordination potential after the metal complex is coordinated to the substrate. This requires that the excess metal complex be washed off before competition with competing agent. If the metal complex was not added in excess to the available coordination sites of the substrate, the metal complex will exhaust all coordinating sites in binding the substrate and no competition occurs. In the case of porous membranes, it also leads to blockages of the pores. Controlling excess is especially difficult when only a part of a membranes needs to be treated such as in stripping membranes with affinity agent. To this end, Example 3 and 4 disclosed in Method of Controlled Competitive Exchange, exemplifies acetate (first agent) capped metal complexes bound to a nitrocellulose membrane (i.e. substrate) and exchanged with streptavidin (Example 3) or an antibody (Example 4). Whereas in this application a mixture of metal complex and affinity agent is stripped onto a membrane. If the affinity agent is the first agent on the substrate there is no competition by a competing agent. Once formed, a metal complex-affinity agent complex, i.e. an affinity agent cluster, is a stable complex and cannot be used in competitive exchanged as is described in Method of Controlled Competitive Exchange. In other words, this invention describes the formation of affinity agent clusters, i.e., interconnected affinity networks, in solution by mixing an affinity agent with suitable modified metal complexes. The applicant has unexpectedly found that certain modified metal complexes can control the size and required time to form affinity agent clusters. If this cluster formation were prepared in the presence of porous substrates, there would be the potential to form an interpenetrating network of affinity agent clusters through multiple pores of a porous membrane. Such interpenetrating networks may not interact with the substrate by dative covalent bonds or depending on the type of porous membrane, there is also the possibility of the affinity agent network coordinating with some points of the substrate. The possibility that porous membranes could be stripped with a mixture of modified metal complex and affinity agents to form such complex structures and that such interpenetrating affinity agent clusters were still functional is unknown in the prior art.

In embodiments, the affinity agent used to assess the reduced reactivity by comparison to that with an unmodified oligomeric metal coordination complex is an antibody such as an IgG antibody including monoclonal mouse antibody against virus antigens such as Flu and SARS-COV-2 and polyclonal antibodies such as goat anti-mouse antibody.

In embodiments, the reduced reactivity of the modified oligomeric metal coordination complex may be defined as a reduced level of reactivity with an lgG antibody including a GAM IgG antibody or a protein such as BSA as compared with that of a corresponding unmodified metal complex, especially a corresponding fully hydrated metal complex (such a complex has non- or weakly coordination anions as ligands).

In embodiments, the reduced reactivity of the modified oligomeric metal coordination complex may be defined as a reduced level of reactivity with an IgG antibody including a GAM IgG antibody or a protein such as BSA as compared with that of an oxo-bridged chromium (III) complex. In embodiments, the oxo-bridged chromium (III) complex used for comparison purposes may be that as formed in Solution 1 of Example 1 in the Examples section.

In embodiments of any of the aspects described herein, the at least one modified metal coordination complex is a capped metal coordination complex and/or a metal coordination complex formed at a low pH (for example at a pH below 3.8). It will be appreciated that the term ‘modified’ oligomeric metal coordination complex is considered any oligomeric metal coordination complex having capping ligands/groups.

In embodiments, the modified oligomeric metal coordination complex has been modified to display capping groups coordinately bound to the metal of the oligomeric metal coordination complex. The capping groups will alter the reaction kinetics of the now modified oligomeric metal coordination complex with moieties on the at least one affinity agent as they will be more resistant to being displaced (due to their greater avidity or multiplicity of potential ligands on a macromolecule) than, for example, simple counterions. The moieties of the at least one affinity agent will therefore react more slowly and with more affinity agents to form an appropriate interconnected affinity network. This slowing of the coordination between the oligomeric metal coordination complexes and affinity agent allows time for the liquid formulation to be contacted with the porous substrate, or a region thereof, while bonds are still forming and an appropriate interpenetration (such as a SIPN and/or IPN) at the molecular level with the polymer forming the porous substrate may be achieved.

In embodiments, the method may further include the step of selecting or controlling the relative extent of the total coordination capacity of the oligomeric metal coordination complex which is taken up by the capping groups. That is, there may be benefits in choosing or modifying the % of the total coordination capacity of the metal ions of the oligomeric metal coordination taken up by capping groups (as measured by that remaining following formation of the oligomeric metal coordination complex itself—as a coordination interaction is reversible, this percentage is the starting percentage taken up by the capping groups). For example, the % of the total coordination capacity taken up by capping groups may be greater than 10%, or 20% or 30% or 40% or 50% any of which values may be combined to form a range with a maximum value of less than 600%, 400%, 200% or 100%. Where the capping groups are in excess of the available coordination potential of the oligomeric metal complex, this excess leads to greater competition for coordination to the available oligomeric metal complex. In this situation, the degree of excess also changes the reaction kinetics of the now modified oligomeric metal coordination complex with the at least one affinity agent as there are more capping groups in competition.

Appropriate capping groups will therefore be those which slow down coordination of the modified oligomeric metal coordination complexes with the affinity agent but do not prevent it. Without this control, such as in the approach of binding biomolecules using standard unmodified oligomeric metal coordination complexes, the metal complexes will simply form tightly bound clusters with the affinity agent and will not provide for appropriate functionality of said affinity agent. The displacement of the capping groups should occur over an appropriate commercial timeframe which can be easily tested by running parallel reactions of oligomeric metal coordination complexes modified with different capping agents and exposed to the same affinity agent.

In embodiments, useful capping groups may be those that include nitrogen, oxygen, or sulphur as dative bond forming groups. More preferably, the dative bond forming groups of the capping agent are oxygen or nitrogen. Even more preferably, the capping agent is one comprising a dative bond forming group which is an oxygen containing group.

In embodiments, the oxygen containing group of the capping group is selected from the group consisting of sulphates, phosphates, carboxylates, sulphonic acids and phosphonic acids.

In embodiments, the capping group may be selected from the group consisting of formate, acetate, propionate, oxalate, malonate, succinate, maleate, sulphate, phosphate, and hydroxyacetate. In embodiments, the capping group may be selected from the group consisting of formate, acetate, propionate, oxalate, malonate, succinate, maleate, citrate, sulphate, phosphate, an amino acid, naphthalene acetate, and hydroxyacetate.

In embodiments, the capping group may be selected from the group consisting of formate, propionate, oxalate, malonate, succinate, glutarate, maleate, citrate, aconitate, sulphate, phosphate, and hydroxyacetate. In embodiments, the capping group may be selected from the group consisting of formate, acetate, propionate, oxalate, malonate, succinate, maleate, citrate, sulphate, phosphate, an amino acid, naphthalene acetate, and hydroxyacetate. In embodiments, the capping group may be selected from the group consisting of oxalate, malonate, succinate, glutarate, adipate, maleate, citrate, and aconitate. In embodiments, the capping group may be selected from the group consisting of formate, acetate, propionate, oxalate, malonate, succinate, glutarate, maleate, citrate, and aconitate. For example, the capping group may be selected from the group consisting of acetate, oxalate, malonate, succinate, and citrate. In one example, the capping group may be selected from the group consisting of acetate, oxalate, phosphate and succinate. In a preferred example, the capping group may be selected from the group consisting of acetate, oxalate, and succinate. The capping group may be oxalate. The capping group may be succinate.

In embodiments, the capping group may be carboxylate or phosphate, preferably carboxylate. For example, the carboxylate may be a dicarboxylate or a tricarboxylate, preferably a dicarboxylate.

In embodiments, the capping group is a monodentate, bidentate or multidentate capping agent. In embodiments, the capping group is a monodentate or bidentate capping agent.

In embodiments, the capping group may be a component of a buffer solution including a phosphate buffer solution.

In embodiments, the capping group may have a lower molecular mass and/or lower coordination strength for the oligomeric metal coordination complex and/or lower electron density and/or fewer number of ligand binding sites than the at least one affinity agent which will displace it.

In embodiments, the capping group has a molecular mass of less than 1000 Daltons, or less than 500 Daltons, or less than 400 Daltons, or less than 300 Daltons. Any of these values may be combined with a lower value of 10, 30 or 50 Daltons to form a range of molecular mass values for the capping agent such as 10 to 1000, 10 to 500, 10 to 400 or 10 to 300 Daltons.

In embodiments, the capping group has a molecular mass of less than 1000 Daltons, or less than 500 Daltons, or less than 400 Daltons, or less than 300 Daltons. Any of these values may be combined with a lower value of 10, 30 or 50 Daltons to form a range of molecular mass values for the capping agent such as 10 to 1000, 10 to 500, 10 to 400 or 10 to 300 Daltons.

In embodiments, the capping group is not simply a counterion of the oligomeric metal coordination complex or a group donated by a base. For example, in forming oligomeric metal coordination complexes it is common to expose the metal complex to a base, such as ethylene diamine, which simply encourage formation of the desired complexes. While the amine nitrogen may be, to a small degree, incorporated into the formed oligomeric metal coordination complex it does not have a significant enough effect on the subsequent reactivity of the oligomeric metal coordination complex to be considered a capping group. Therefore, in one embodiment, the capping group is not one donated by a base, including ethylene diamine.

In embodiments, the capping group is a coordinating capping group. That is, the capping group forms at least one coordinate bond with the oligomeric metal coordination complex.

In embodiments, the modified oligomeric metal coordination complex has been modified to form an oligomeric complex at a pH below 5.0. The inventors have surprisingly found that there is a complex relationship between the size of the formed oligomeric complex, the type of capping group and the excess used, and pH of the oligomeric metal coordination complex solution which results in complexes that demonstrate a modified reactivity to the subsequently introduced affinity agent. While not wishing to be bound by theory, at higher pH values such as above pH 5.0, the strength of binding of metal complexes to affinity agents progressively grows stronger. Further, their ability to strongly react with any other ligand such as a polymer of the porous substrate also becomes stronger. Higher pH conditions of the oligomeric metal coordination complexes resulted in less functional clusters. At lower pH values, such as below pH 5.0, the reactivity of the oligomeric metal coordination complexes decreases and whether augmented by capping groups of certain binding strength and excess, and whether buffered by the capping group to help stabilise some target pH, allows fine control that allows the formation of the interconnected affinity network in the presence of the porous substrate, as described.

In embodiments, the modified oligomeric metal coordination complex has been modified by formation at a pH below 5.0, or below 4.5, or below 4.0, or below 3.8, or below 3.7, or below 3.6, or below 3.5 or below 3.4 or below 3.3 or below 3.2 or below 3.1 or at or below 3.0. The pH at formation will, in combination with all instances of the above cited upper limits, be greater than 1.0.

This pH may be the final pH when the metal coordination complex is considered to have formed. This is because many metal salts, such as chromium salts, are highly acidic and release hydrogen ions as the complexes form. The pH of such a solution can therefore become more acidic over time as the complexes form and it is the final pH which is key to the nature of the metal coordination complex formed, and so, its degree of modification.

Therefore, in embodiments, the method may further comprise the step of forming a modified oligomeric metal coordination complex. The forming may be a modification of an existing oligomeric metal coordination complex or it may be concurrent formation of the oligomeric metal coordination complex and modification of same as it forms.

The step of forming the modified oligomeric metal coordination complex may include contacting the oligomeric metal coordination complex with a solution comprising a capping group. Alternatively, the step of forming the modified oligomeric metal coordination complex may include exposing the corresponding monomeric metal coordination complex to a base to thereby form the modified oligomeric metal coordination complex at a pH below 5.0. Alternatively, the step of forming the modified oligomeric metal coordination complex may include reaction of the corresponding monomeric metal coordination complex with capping groups and a base in organic solvents with elevated temperature.

The method may further include the step of adjusting the pH of the liquid formulation, comprising the modified metal coordination complex, to be between pH 1.5 to pH 5.0 and/or controlling the temperature of the liquid formulation to be between 15 to 30° C.

In embodiments, the step of adjusting the pH may include adjusting the pH of the solution in which the oligomeric metal coordination complexes are forming to ensure the desired degree of modification. This may comprise allowing the pH to become more acidic due to the release of hydrogen ions by the metal salts employed or it may comprise the addition of a base, such as ethylene diamine or a metal hydroxide, to mop up some of the released hydrogen ions to prevent the solution becoming too acidic. If a base is added then the amount will be such that the solution is still acidic, as defined above.

In embodiments, the modified metal coordination complex can be formed via the direct reduction of chromium (VI) oxide in the presence of suitable capping groups such as acetic acid. Once the complex is synthesised, the pH can be adjusted as required.

It will also be appreciated that, in embodiments, the modified oligomeric metal coordination complex may include various capping groups having on/off rates which are appreciably slower than pre-existing water and other ligand groups, and hence will affect coordination with an additional component of the formulation.

The oligomeric metal coordination complex will be discussed below, in terms of available variations in the synthetic approach and the potential for differences thereby achieved in the final product.

The oligomeric metal coordination complexes can be formed by providing conditions for forming electron donating groups for bridging or otherwise linking or bonding two or more metal ions. When not already commercially available, this can be done by providing a pH above pH 1, and preferably between about 1 to 5, or about 2 to 5 to the solution when forming the complexes. Clearly, the chosen pH will depend on the approach by which modification of the oligomeric metal coordination complex is to be achieved. That is, while pHs above 3.8 may be appropriate for forming the oligomeric metal coordination complex when they are to be modified by use of capping groups, a pH below 3.8 is highly desirable for the oligomeric metal coordination complexes formed in aqueous solutions. In non-aqueous solutions, pH cannot be used as an appropriate measure and so metal coordination complexes to be formed are determined by the amount of base/acid in the organic reaction solvent.

Various chromium salts such as chromium chloride, chromium nitrate, chromium sulphate, chromium acetate, chromium perchlorates, may be used to form a chromium-based oligomeric metal coordination complex. Unless pre-existing in some oligomeric form and used ‘as is’, these salts are mixed with an alkaline solution, such as sodium hydroxide, potassium hydroxide, lithium hydroxide, sodium bicarbonate, sodium sulphite and ammonium hydroxide to form different metal coordination complexes. Organic reagents that can act as bases such as ethylene diamine, bis(3-aminopropyl) diethylamine, pyridine, imidazoles, can also be used. The size and structure of the oligomeric metal coordination complex can vary with pH, temperature, choice of solvent and other conditions.

In embodiments, the liquid carrier forming the liquid formulation with the oligomeric metal coordination complex associated with at least one affinity agent may be an aqueous carrier.

In one embodiment, the method further includes the step of at least partially removing the liquid carrier from the porous substrate. The removal of the liquid carrier may comprise an active heating step or may occur simply by maintaining the binding support at room temperature in a humidity-controlled environment.

The step (c) of contacting the polymeric porous substrate with the liquid formulation may be performed within a predetermined period of time after the modified oligomeric metal coordination complexes, are first contacted with the affinity agent.

In embodiments, the predetermined time is less than the time taken for the bonding between the oligomeric complexes, modified or otherwise, and the at least one affinity agent to be 20% complete, or less than 30% complete, or less than 40% complete, or less than 50% complete, or less than 60% complete, or less than 70% complete, or less than 80% complete, or less than 90% complete.

Viewed another way, the predetermined time is less than the time taken for the interconnected affinity network to be 20% complete, 30% complete, 40% complete, 50% complete, 60% complete, 70% complete, 80% complete, or 90% complete.

In embodiments, the predetermined period of time may be less than 10 hours, less than 9 hours, less than 8 hours, less than 7 hours, less than 6 hours, less than 5 hours, less than 4 hours, less than 3 hours, less than 2 hours, less than 1 hour, less than 55 minutes, less than 50 minutes, less than 45 minutes, less than 40 minutes, less than 35 minutes, less than 30 minutes, less than 25 minutes, less than 20 minutes, less than 15 minutes, less than 10 minutes or less than about 5 minutes.

As discussed, the predetermined time is one important component of controlling the extent of bonding between the oligomeric metal coordination complexes and affinity agent prior to exposure to the porous substrate thereby controlling the extent to which the interconnected affinity network may form while interpenetrating the polymeric porous substrate. It will be appreciated that the selection of the predetermined time will be influenced by the modification of the oligomeric metal coordination complexes. For example, if the oligomeric metal coordination complexes are modified by pH, capping groups or other means such that their reactivity is significantly reduced then the selected predetermined time may be longer than if the oligomeric metal coordination complexes are only moderately modified and so would react faster thereby necessitating a slightly shorter predetermined time period.

Temperature will also affect the time taken to substantially complete the interconnected affinity network and so the predetermined time may be controlled by a step of controlling the temperature. In embodiments, the temperature may be controlled to be greater than 20° C., greater than 25° C., greater than 30° C., or greater than 35° C. For example, the temperature may be controlled to be about 37° C.

The step of contacting the polymeric porous substrate with the liquid formulation may be a step of striping the porous substrate when the binding support is for use as a test strip in an assay, such as a LFA. In such embodiments, the contacting will only occur on portions of the binding support to form test and/or control lines for the assay.

Striping of the test and control lines onto a porous substrate, such as a nitrocellulose membrane, is well known in the art with a number of commercially available dispensing instruments available. Non-contact dispensing such as spray or jetting typically requires less volume to stripe, but can result in greater run-to-run variability. Contact dispensing systems on the other hand have relatively low run-to-run variability, but require additional volume in order to stripe the same amount of material. The choice of the striping approach and the specifics of the liquid formulation to allow for effective striping are well understood in the art and are easily tailored to provide the appropriate width of striped area, reagent density etc.

In a third aspect of the disclosure, there is provided a method of capturing a target molecule from a sample including the steps of:
(a) providing a binding support comprising a polymeric porous substrate formed from at least one polymer; an oligomeric metal coordination complex associated with the at least one polymer forming the polymeric porous substrate; and at least one affinity agent specific for the target molecule associated with the oligomeric metal coordination complex; and
(b) contacting the binding support with the sample comprising the target molecule, to thereby capture the target molecule from the sample.

In a fourth aspect of the disclosure, there is provided an assay comprising:
(a) contacting a binding support with a target molecule, the binding support comprising:
   (i) a polymeric porous substrate formed from at least one polymer;
   (ii) an oligomeric metal coordination complex associated with the at least one polymer forming the polymeric porous substrate;
   (iii) at least one affinity agent specific for the target molecule associated with the oligomeric metal coordination complex; and
(b) detecting the binding of the target molecule with the target-specific affinity agent.

The binding support, polymeric porous substrate, oligomeric metal coordination complex and at least one affinity agent may be as described for any embodiment, or combination of embodiments, of the first aspect.

In embodiments of the third and/or fourth aspect, the coated substrate may be useful for detecting target molecules in samples such as biological samples, diagnostic samples, food samples, air samples, water samples and the like. Non-limiting examples of biological and/or diagnostic samples include tissue, organ and tumour biopsies, body fluids such as urine, blood, serum, cerebrospinal fluid, semen, tears and sputum, although without limitation thereto

In embodiments, the assay is an immunoassay.

In certain embodiments, the assay is a lateral flow assay.

### EXAMPLES

### Example 1: Preparation of Metal Coordination Complex Solutions

Examples of oligomeric metal coordination complexes are described. Depending on the metal ion, salt, the base, the final pH and other ligands used, the metal coordination complex solutions exhibit different binding properties.

### Solution 1

In this example, chromium perchlorate hexahydrate (45.9 g) was dissolved into 480 mL of purified water and mixed thoroughly until all solid dissolved. Similarly, 8 mL of ethylene diamine (EDA) solution was added to 490 mL of purified water. The solutions were combined by the dropwise addition of the EDA solution into the chromium salt solution and stirred overnight at room temperature, and then left to equilibrate to a pH of approximately 4.5.

### Solution 2

Similar to Solution 1, different ratios of chromium perchlorate and ethylenediamine solution can be used to generate solutions having a different pH such as pH 3.0, pH 4.0, pH 5.0 or some other pH. As an example, chromium perchlorate hexahydrate (103.5 g) was dissolved into 1000 mL of purified water and mixed thoroughly until all solid dissolved. 8 mL of ethylene diamine solution was added to 1000 mL of purified water. The solutions were combined by the dropwise addition of the EDA solution into the chromium salt solution, and stirred overnight at room temperature, and then left to equilibrate to the desired pH.

### Solution 3

In this example, 500 ml 100 mM acetate buffer at pH 3.6 is added dropwise to 500 ml of Solution 2 with stirring, and then left to equilibrate to a pH of approximately 3.0 to form Solution 3A. Depending on the pH of the respective solutions and concentration of acetate buffer, different versions of Solution 3 can be formed. One example (Solution 3B) is when double the amount of acetate is added. Even the method of synthesis (to form hydroxo and oxo bridging) can form different versions of Solution 3 having different acetate ligands coordination strengths to the metal ion of the complex. It is not necessary for the acetate ligand to be all coordinated to the metal complex and it will be appreciated that more than one type of ligand comprising the buffer can also be used.

### Solution 4

In this example, 500 ml 100 mM oxalic acid buffer at pH 3.5 is added dropwise to 500 ml of Solution 2 with stirring, and then left to equilibrate to a pH of 3.0 to 3.5. Depending on the pH of the respective solutions and concentration of oxalate acid buffer, different versions of Solution 4 can be formed. This can be considered another representative modified oligomeric metal coordination complex.

### Solution 5

Similar to Solution 4, alternative excesses of oxalic acid can be used to generate solutions having a far larger excess of oxalic acid to the metal complex of Solution 2. As an example, using 200 mM of oxalic acid buffer will produce a metal ion to oxalic acid ratio of 1:2 (Solution 5A), and by using more dilute mixtures, even higher oxalic acid excesses can be added to give metal ion to oxalic acid ratios of 1:4 (Solution 5B). Metal complex solutions with many other different metal ions to ligand ratios can be formed.

### Solution 6

In this example, 500 ml 100 mM succinic acid buffer at pH 3.5 is added dropwise to 500 ml of Solution 2 with stirring, and then left to equilibrate to a pH of 3.0 to 3.5. This can be considered another representative modified oligomeric metal coordination complex. Similar to Solution 4 and 5, different ratios of succinic acid to Solution 2 can be used to generate solutions having a far larger excess of succinic acid to the metal complex of Solution 2. Such examples are metal ion to succinic acid ratio of 1:1 (Solution 6A), ratio of 1:2 (Solution 6B) and ratio of 1:4 (Solution 6C). Many different ligands, combinations and ratios can be formed.

### Example 2: Clustering with Oligomeric Metal Complexes Using BSA Model

### Example 2a

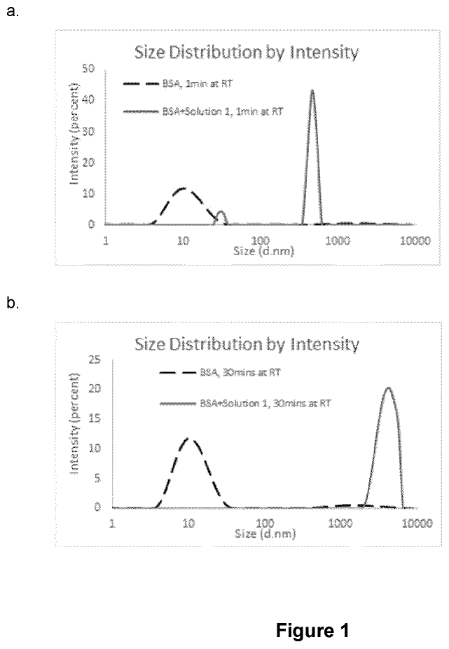

In this example, 8 mg/ml of Bovine Serum Albumin (BSA) was dissolved in 25 mM MES buffer at pH 6.0. To 500 μL BSA (final conc of 4 mg/ml) samples, 500 μL of Solution 1 having a final concentration of 4 mM were mixed. Samples were transferred into a zeta potential cuvette for measurement on a Malvern Nano ZS Zetasizer over a 60 min time period. As shown FIG. 1, Solution 1 complex (unmodified) was highly reactive resulting in rapid protein aggregation. Within 30 mins, large insoluble (cloudy suspension) aggregates ranging in size between 2 to over 8 microns (average of approximately 4 microns) were formed. Diluting the Solution 1 complex to 2 mM still gave cloudy solutions with similar size aggregates as before. However, smaller clusters in the 200 to 700 nm range also remained (data not shown). Decreasing the amount of unmodified metal complexes led to a mixture of heterogenous sized clusters of which the majority existed as insoluble aggregates.

### Example 2b

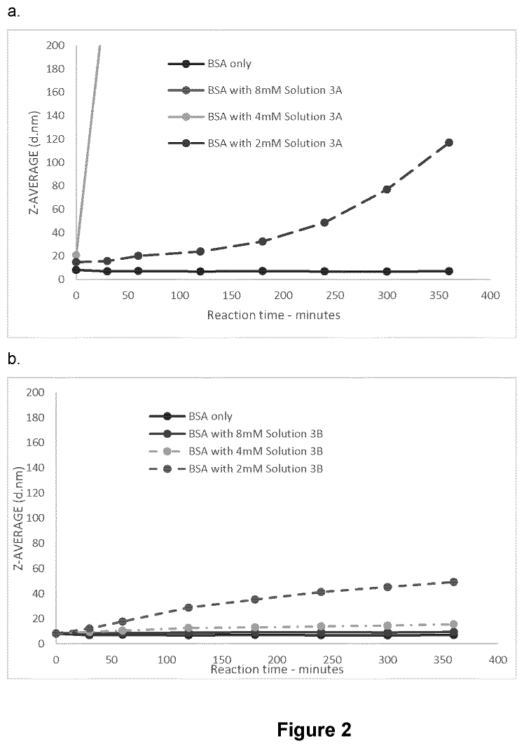

Using the same procedure as in Example 2a, the characteristics of Solution 3A and 3B (modified by acetate capping ligands) at 2, 4 and 8 mM concentrations were assessed. As shown FIG. 2a, Solution 3A complex still formed large aggregates using the higher concentrations. But at 2 mM, the reaction has slowed to give cluster sizes of around 110 nm after 7 hrs. With Solution 3B (FIG. 2b) at a concentration of 2 nM clusters of around 50 nm after 7 hours were observed.

### Example 2c

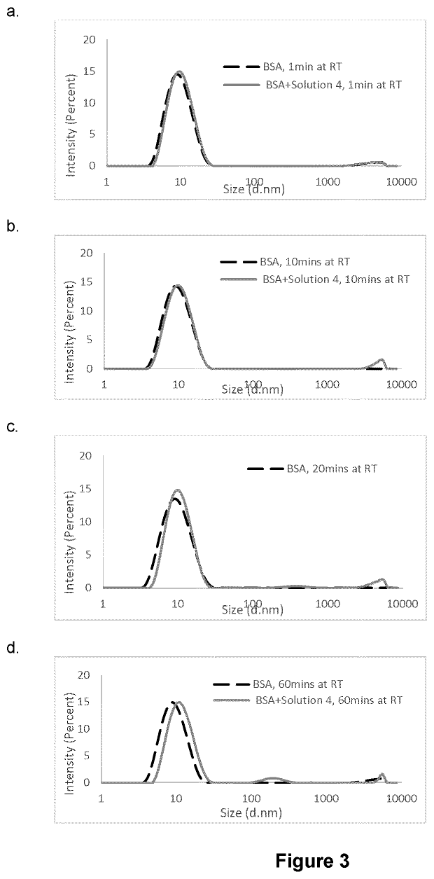

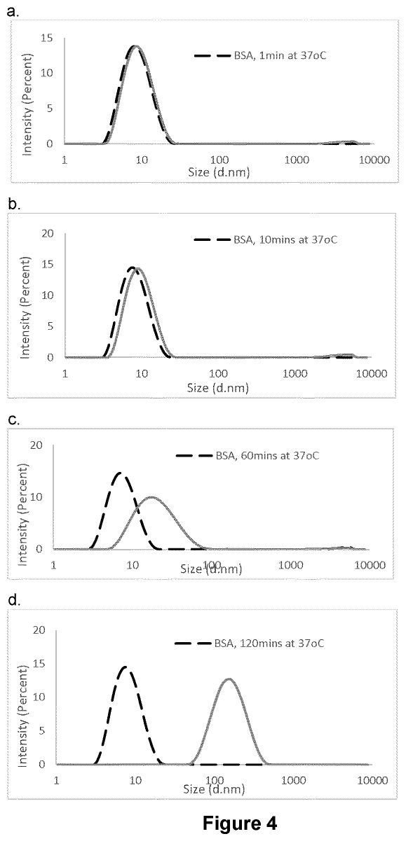

Using the same procedure as before, the effect of 4 mM Solution 4 (modified via oxalic acid capping ligand) was tracked over 60 mins at room temperature. As shown in FIG. 3, only a small amount of cluster formation was observed. However, if the same reaction is performed at 37° C. over 120 min (FIG. 4), small uniform clusters of BSA are being formed. By controlling the protein:metal complex ratios, buffer and temperature conditions, it is possible to form uniform protein clusters.

### Example 2d

The characteristics of the oxalate modified metal complexes at different concentrations and ratios were further assessed for forming controlled protein clusters.

As shown in the example, metal complexes having three different oxalic acid capping ratios were compared, i.e., metal complex to oxalic acid 1:1 (Solution 4), 1:2 (Solution 5A) and 1:4 (Solution 5B). The different BSA/metal complex mixtures were compared with BSA/water mixture (Control) over a 7-hour period.

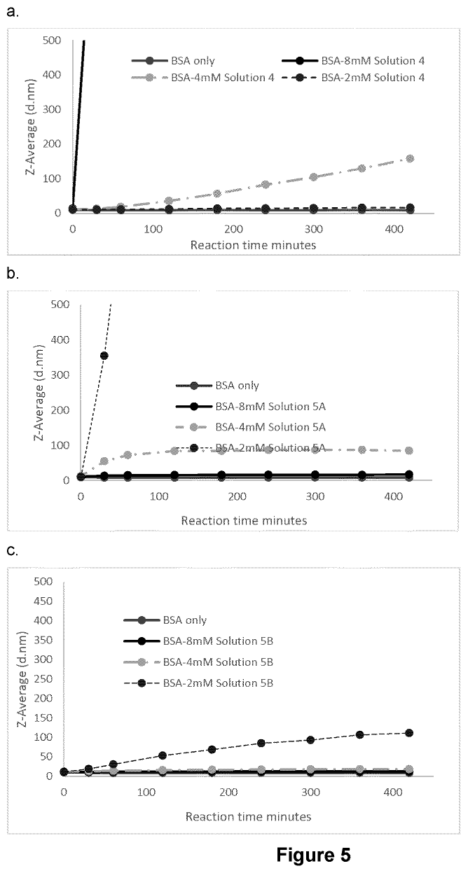

FIG. 5a shows the rate of BSA clusters (in nanometers) being formed with different dilutions of Solution 4. Solution 4 at 4 mM showed a steady increase in BSA cluster size stabilising around 140 nm after 7 hrs.

FIG. 5b shows the result of a similar experiment using a metal complex to oxalic acid ratio of 1:2 (Solution 5A) and FIG. 5c shows the rate using a metal complex to oxalic acid ratio of 1:4 (Solution 5B). Solution 5A at 2 mM provided the largest clusters stabilising around 800 nm after 7 hrs. Solution 5B at 2 mM concentration also gave the largest clusters, which was stabilizing around 100 nm after 7 hrs.

### Example 2e

Using the same procedure as above, the effect of different capping agents is compared under similar conditions.

Table 1 shows the trends with metal complex to oxalic acid ratio of 1:4 (Solution 5B) and Table 2 shows the trends with metal complex to succinic acid ratio of 1:4 (Solution 6C).

As shown, the rate of BSA cluster formation is different and changes with not only the capping agent but also the ratio between the protein and the modified oligomeric metal complex.

TABLE 1
The zeta size in nanometres of the Bovine
Serum Albumin (BSA) clusters formed using oxalic
acid capped (Solution 5B) at different ratios to BSA.

| Reaction | BSA | BSA-8 mM | BSA-4 mM | BSA-2 mM |
|---|---|---|---|---|
| time mins | only | Solution 5B | Solution 5B | Solution 5B |
| 0 | 10 | 11 | 11 | 11 |
| 30 | 9 | 11 | 13 | 19 |
| 60 | 8 | 11 | 14 | 30 |
| 120 | 9 | 12 | 16 | 53 |
| 180 | 9 | 13 | 17 | 68 |
| 240 | 9 | 13 | 17 | 84 |
| 300 | 9 | 12 | 18 | 93 |
| 360 | 9 | 13 | 18 | 106 |

TABLE 2
The zeta size in nanometres of the Bovine Serum
Albumin (BSA) clusters formed using succinic acid
capped (Solution 6C) at different ratios to BSA.

| Reaction | BSA | BSA-8 mM | BSA-4 mM | BSA-2 mM |
|---|---|---|---|---|
| time mins | only | Soln 5B | Soln 5B | Soln 5B |
| 0 | 8 | 9 | 139 | 999 |
| 30 | 7 | 15 | 578 | 4416 |
| 60 | 7 | 9 | 668 | — |
| 120 | 8 | 10 | 562 | — |
| 180 | 9 | 11 | 559 | — |
| 240 | 8 | 11 | 553 | — |
| 300 | 8 | 12 | 589 | — |
| 360 | 8 | 12 | 507 | — |

As shown, the capping groups allow fine control over the rate of protein cluster formation and its final size. The use of these methods to form affinity agent networks within porous substrates was investigated in the following.

### Example 3: Comparison of Antibody Cluster Vs Non-Cluster at Different pHs (Control)

A lateral flow test strip was used to exemplify the present invention. A conventional lateral flow strip comprises an analyte application zone, a detection zone where analytes are captured by ligands fixed on porous substrate and a wicking system that draws analyte across capture ligand by capillary action and then retains overflow solution. Analyte bound to the capture ligand is commonly detected using nanoparticles or other known reporter systems.

In this example, a direct immunoassay lateral assay model was employed. A mouse monoclonal antibody for FluA (Cat #CO1736, Meridian) was conjugated to a nanoparticle (detection moiety) and captured with the ligand, goat anti-mouse antibody (Lampire), striped on a nitrocellulose membrane on a plastic support (HF090 card HF090MC100, Millipore). The controlled application was executed using the Linomat V (CAMAG) at a volume of 1 μL/cm. After striping, the ligand membrane was dried for 2 hours at 37° C. in a fan forced incubator. The membrane was then stored in a sealed foil pouch with desiccant until use.

The Control for this example is passive immobilization of ligand onto nitrocellulose membrane. The GAM antibody in Carbonate buffer, pH 8.5 gave the best results so this was used as the benchmark for comparison purposes.

The analyte/detection moiety, mouse IgG antibody, was conjugated to Europium nanoparticles (Merck Ref #F1-Eu-030) activated with Solution 1. In brief, Europium-chelate latex particles were separated from solution by centrifugation and then the particles were redispersed in Solution 1 to a concentration of 2 mg/mL. Particles were then separated from Solution 1 by centrifugation and washed twice with DI water. 0.04 μg of mouse IgG conjugate was used for each strip. Analyte bound to the capture ligand was detected using a fluorescent reader (Axxin).

The goat anti-mouse antibody was immobilised onto the membrane in the presence of oxalate capped metal coordination complexes (Solution 4). In brief, 25 μl of Goat anti-Mouse antibody (0.5 mg/ml) in 3 different striping buffers (MES buffer, pH 5.2 and pH 6.0, and Carbonate buffer, pH 8.5) were vortexed and mixed with 25 μl of both 0.2 and 1.0 mM of Solution 4 for 20 mins to form a final 0.25 mg/ml antibody in 0.1- and 0.5-mM

Solution 3. Approximately 60 mins later the mixture was striped onto the membrane. The lateral flow was then assembled and tested as described above.

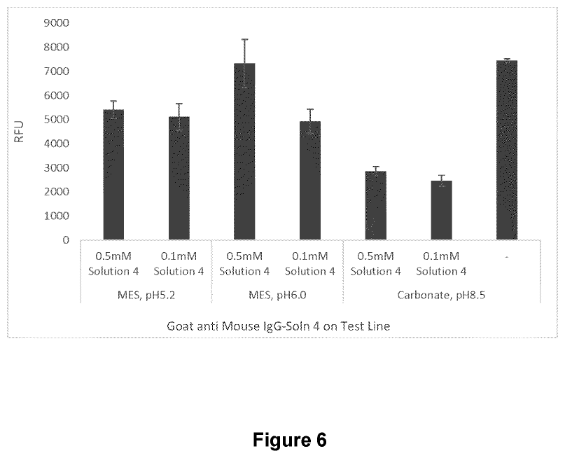

Stable immobilization of GAM antibody was achieved at all pH values but the condition using 0.5 mM Solution 4 at pH 6.0 was found to give the largest signal (FIG. 6).

### Example 4: Comparison of Antibody Cluster Vs Non-Cluster:Striping at Higher Concentration

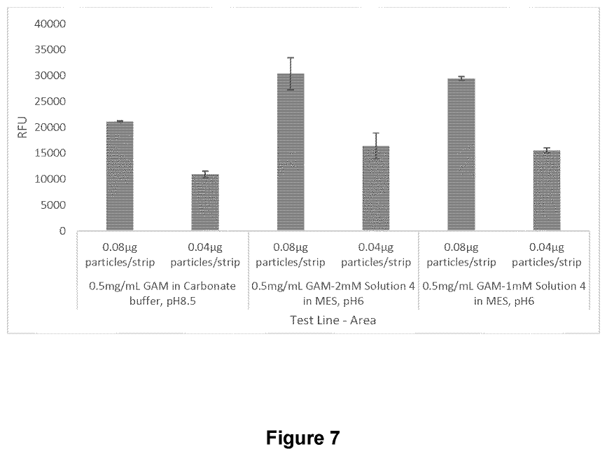

In this example, the Antibody and Solution 4 were mixed and striped onto the membrane between 10 and 20 mins. In brief, 25 μl of Goat anti-Mouse antibody (1.0 mg/ml) in MES buffer at pH 6.0 was mixed with 25 μl of both 2.0 and 4.0 mM of Solution 3 to form a final 0.5 mg/ml antibody in 1.0- and 2.0 mM Solution 4. This was quickly striped onto the membrane and compared to passive binding of 0.5 mg/ml antibody in Carbonate buffer, pH 8.5) as previously described in Example 3. In this case, two conjugate loadings of 0.04 μg (Control) and 0.08 μg were also compared. All other conditions were the same as in the previous Example. This example shows a clear increase (approx. 50%) in signal compared to passive (FIG. 7).

### Example 5: Time Dependent Striping of Antibody/Metal Complex Clusters

In this example, the Antibody and Solution 4 were mixed and striped onto the membrane between 10 and 20 mins, mixed and striped 1.5 hrs later and also mixed and striped 3 hrs later. In brief, 25 μl of Goat anti-Mouse antibody (1.0 mg/ml) in MES buffer at pH 6.0 was mixed with 25 μl of both 2.0 and 4.0 mM of Solution 4 to form a final 0.5 mg/ml antibody in 1.0- and 2.0 mM Solution 4. After leaving the mixture for different times, the mixture was striped onto the membrane and compared to passive binding of 0.5 mg/ml antibody in Carbonate buffer, pH 8.5) as previously described in Example 3. In this experiment, there was no difference in assay outcome with different mixing times.

### Example 6: Striping Antibody/Metal Complex Clusters to Blocked Membranes

In this example, the Antibody and Solution 4 were mixed and quickly striped onto the membrane (10 to 20 mins). The procedure is as described in Example 4 but, in this example, the membrane was ‘pre-blocked’ using 5 mg/ml BSA (A3070, Sigma) in either MES pH 6.0 or Carbonate pH 8.5 (for the Control) buffer. As expected, the signal for the Control was depressed compared to using non-blocked membranes but there was still partial binding. In comparison, certain antibody/metal complex mixtures gave almost 3× increase in signal when compared to the Control (FIG. 9). While passive binding could not be eliminated, there is enhanced binding in the case of antibody clusters when compared to using non-blocked membranes shown in FIG. 7.

### Example 7. Striping Antibody/Metal Complex Clusters Formed Using Alternative Capping Agent

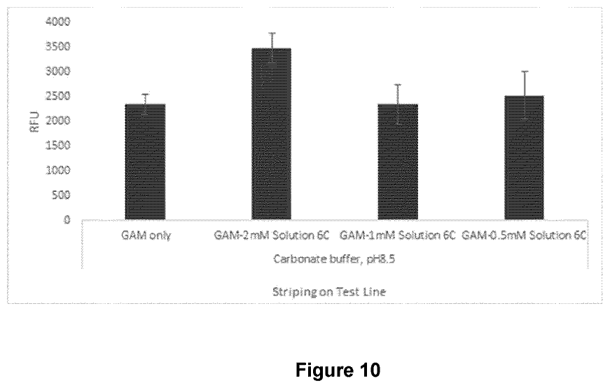

In this example, the Antibody and Solution 6C were mixed and striped onto the membrane (10 to 20 mins). The procedure is as described in Example 4 except a different buffer and metal complex was used. In brief, 25 μl of Goat anti-Mouse antibody (1.0 mg/ml) in Carbonate buffer at pH 8.5. was mixed with 25 μl of both 1.0, 2.0 and 4.0 mM of Solution 6C to form a final 0.5 mg/ml antibody in 0.5-, 1.0- and 2.0 mM Solution 6C. This was striped onto the membrane and compared to passive binding of 0.5 mg/ml antibody in Carbonate buffer, pH 8.5. In comparison to the Control using Carbonate Buffer as well as the clusters formed in MES buffer (Example 4), the clusters formed in Carbonate Buffer gave even better results in the case of 2 mM Solution 6C (FIG. 10).

### Example 8: Comparison of Antibody/Metal Complex Clusters in a Sandwich Assay

A lateral flow half test strip was used to compare any differences in a FluA sandwich assay model. In brief, a mouse monoclonal antibody for FluA (Cat #10-150H, Fitzgerald) was conjugated to Europium nanoparticles (Merck Ref #F1-Eu-030) as the detection moiety and captured on two test lines comprising an anti-FluA antibody (Cat #7304, Medix) and an antiFluB antibody (Cat #9906, Medix) diluted to 0.5 mg/mL in carbonate buffer striped onto nitrocellulose membrane on a plastic support (HF090 card HF090MC100, Millipore) using the Linomat V (CAMAG). The membrane was blocked with 5 mg/ml BSA in carbonate buffer prior to striping the test lines. After striping, the ligand membrane was dried for 2 hours at 37° C. in a fan forced incubator. The membrane was then stored in a sealed foil pouch with desiccant until use.

Three different mAb clusters were formed on the test line using 0.5 mg/ml FluA mAb with 0.5 mM, 1 mM and 2 mM of Solution 6C, respectively.

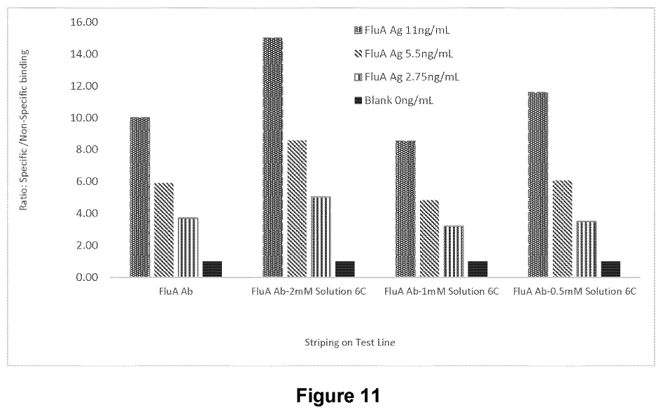

The Flu A antigen was used at 3 different concentrations; 2.75, 5.5 and 11 ng/ml in PBS, 1% BSA, pH7.4, and Blank was also included. The stock 10 mg/ml of conjugate particles were diluted to 2 μg/mL in Lysis buffer (Tris buffer pH8, containing N-lauroyl sarcosine sodium salt, L-aspartic acid, Tergitol, sodium dodecyl sulfate sodium deoxycholate and sodium azide) and 25 μL was used for each strip. Antigen capture was detected using a fluorescent reader (Axxin). As shown in Table 3 and FIG. 11, the antibody cluster formed with Solution 6C, had different outcomes indicating that the functional activity of antibodies in the clusters were different. As shown in Table 3, the clusters gave low background signals and low cross reactivity to the Flu B antigen compared to the Control. In the case of the cluster formed with 2 mM Solution 6C, there was significant improvement in Sn/S0. The clear difference in performance between the clusters and Control indicate that the functional activity can be manipulated by the amount and type of modified metal coordination complexes.

TABLE 3
Flu A sandwich assays using antibody clusters formed
using Solution 6C compared to Control.

| | | AVERAGE | | | |
|---|---|---|---|---|---|
| | | | FluA Ab | FluA Ab | FluA Ab |
| | | | with | with | with |
| | | | 2 mM | 1 mM | 0.5 mM |
| | | FluA | Solution | Solution | Solution |
| Strip—Test Line | | Ab | 6C | 6C | 6C |
| FluA Ag | 11 ng/mL | 1683 | 700 | 583 | 753 |
| | 5.5 ng/mL | 993 | 400 | 330 | 393 |
| | 2.75 ng/mL | 623 | 235 | 190 | 227 |
| FluB Ag | 42 ng/mL | 157 | 63 | 67 | 50 |
| | 21 ng/mL | 140 | 70 | 83 | 57 |
| | 10.5 ng/mL | 175 | 77 | 73 | 40 |
| Blank | 0 ng/mL | 168 | 47 | 68 | 65 |
| | | Ratio: Sn/S0 | | | |
| | | | FluA Ab | FluA Ab | FluA Ab |
| | | | with | with | with |
| | | | 2 mM | 1 mM | 0.5 mM |
| | | FluA | Solution | Solution | Solution |
| Strip—Test Line | | Ab | 6C | 6C | 6C |
| FluA Ag | 11 ng/mL | 10.00 | 15.00 | 8.54 | 11.59 |
| | 5.5 ng/mL | 5.90 | 8.57 | 4.83 | 6.05 |
| | 2.75 ng/mL | 3.70 | 5.04 | 2.78 | 3.49 |
| Blank | 0 ng/mL | 1.00 | 1.00 | 1.00 | 1.00 |

1. A method of forming a binding support, the binding support comprising a polymeric porous substrate formed from at least one polymer; an oligomeric metal coordination complex associated with the at least one polymer forming the polymeric porous substrate; and at least one affinity agent associated with the oligomeric metal coordination complex, the method including the steps of:
(a) providing a polymeric porous substrate formed from at least one polymer;
(b) providing a liquid formulation comprising an oligomeric metal coordination complex associated with at least one affinity agent, within a liquid carrier; and
(c) contacting the polymeric porous substrate with the liquid formulation, to thereby form the binding support.

2. The method of forming a binding support of claim 1, wherein the oligomeric metal coordination complex is a modified oligomeric metal coordination complex.

3. The method of forming a binding support of claim 1, wherein the method further comprises the step of forming a modified oligomeric metal coordination complex.

4. The method of forming a binding support of claim 1, wherein the method further includes the step of controlling the reaction pH and/or temperature and/or mixing and/or relative concentrations of modified oligomeric metal coordination complex and/or at least one affinity agent, when the modified oligomeric metal coordination complex and at least one affinity agent are contacted with one another.

5. The method of forming a binding support of claim 1, wherein the modified metal coordination complex is a capped metal coordination complex and/or a metal coordination complex formed at a pH below 5.0.

6. The method of forming a binding support of claim 5, wherein the capping group used to form the capped metal coordination complex is selected from those including one or more of nitrogen, oxygen, or sulphur as dative bond forming groups.

7. The method of forming a binding support of claim 6, wherein the capping group is selected from the group consisting of formate, acetate, propionate, oxalate, malonate, succinate, maleate, sulphate, phosphate, and hydroxyacetate.

8. The method of forming a binding support of claim 5, wherein the at least one modified oligomeric metal coordination complex has been modified by formation at a pH below 3.8.

9. The method of forming a binding support of claim 1, wherein the step of contacting the polymeric porous substrate with the liquid formulation is carried out within a predetermined period of time after the oligomeric metal coordination complex is first contacted with the at least one affinity agent.

10. The method of forming a binding support of claim 9, wherein the predetermined time is less than the time taken for the bonding between the oligomeric metal coordination complexes and the at least one affinity agent to be 20% complete, to less than 90% complete.

11. The method of forming a binding support of claim 9, wherein the predetermined period of time is less than 10 hours, to less than about 5 minutes.

12. A binding support comprising:
(a) a polymeric porous substrate formed from at least one polymer;
(b) an oligomeric metal coordination complex associated with the at least one polymer forming the polymeric porous substrate; and
(c) at least one affinity agent associated with the oligomeric metal coordination complex.

13. The binding support of claim 12, wherein the polymeric porous substrate is formed from one or more natural, semi-synthetic or synthetic polymers, optionally a nitrocellulose including a nitrocellulose membrane.

14. The binding support of claim 12, wherein the oligomeric coordination complex and associated at least one affinity agent form an interconnected affinity network of metal coordination complexes linking affinity agents and at least a portion of the metal coordination complex links at least partially interpenetrating the at least one polymer forming the polymeric porous substrate.

15. The binding support of claim 12, wherein the metal of the metal coordination complex is selected from the group consisting of chromium, ruthenium, iron, cobalt, aluminium, zirconium, and rhodium.

16. The binding support of claim 15, wherein the chromium ion is a chromium (III) ion.

17. The binding support of claim 12, wherein metal coordination complex comprises a ligand having a dative bond forming atom selected from nitrogen, oxygen, or sulfur datively bonded to the metal.

18. The binding support of claim 12, wherein the metal coordination complex is an oxo-bridged chromium (III) complex.

19. The binding support of claim 12, wherein the binding support is a component of an assay device, a filtration system, a chromatographic system, a controlled drug release approach, or a medical device for implantation.

20. A method of capturing a target molecule from a sample including the steps of:
(a) providing a binding support comprising a polymeric porous substrate formed from at least one polymer; an oligomeric metal coordination complex associated with and at least partially interpenetrating the at least one polymer forming the polymeric porous substrate; and at least one affinity agent specific for the target molecule associated with the oligomeric metal coordination complex; and
(b) contacting the binding support with the sample comprising the target molecule, to thereby capture the target molecule from the sample.

21. An assay comprising:
(a) contacting a binding support with a target molecule, the binding support comprising:
(i) a polymeric porous substrate formed from at least one polymer;
(ii) an oligomeric metal coordination complex associated with and at least partially interpenetrating the at least one polymer forming the polymeric porous substrate;
(iii) at least one affinity agent specific for the target molecule associated with the oligomeric metal coordination complex; and
(b) detecting the binding of the target molecule with the target-specific affinity agent.

22. The affinity assay of claim 20, wherein the assay is an immunoassay.

23. The affinity assay of claim 20, wherein the assay is a lateral flow assay.
